(19) United States
(12) Patent Application Publication
(10) Pub. No.: US 2019/0051389 A1
(43) Pub. Date: Feb. 14, 2019

(54) PRIMARY CARE PATIENT PANEL MANAGEMENT

(71) Applicants: Eric Meittunen, Edina, MN (US); Anne Roehrl, Lino Lakes, MN (US)

(72) Inventors: Eric Meittunen, Edina, MN (US); Anne Roehrl, Lino Lakes, MN (US)

(21) Appl. No.: 16/101,116

(22) Filed: Aug. 10, 2018

Related U.S. Application Data
(60) Provisional application No. 62/544,337, filed on Aug. 11, 2017.

(51) Int. Cl. G16H 10/60; G16H 10/20; G16H 50/20; G16H 50/70

(52) U.S. Cl. CPC G16H 10/60; G16H 10/20; G16H 15/00; G16H 50/70; G16H 50/20

(57) ABSTRACT

This specification discloses computer-based systems, methods, devices, and other techniques for managing patient panels for primary care providers. In some implementations, a computing system can be configured to analyze patient data for a large number of patients and primary care providers in a provider organization such as a hospital or clinic.

### CROSS-REFERENCE TO RELATED APPLICATIONS

This application claims priority to U.S. Application Ser. No. 62/544,337, filed on August 11, 2017. The disclosure of the prior application is considered part of the disclosure of this application, and is incorporated in its entirety into this application.

### BACKGROUND

Individuals commonly require medical services to address healthcare needs that may arise, whether expectedly or not, over time. The healthcare system is large and complex, which can make it difficult for patients to identify an appropriate healthcare provider to address their particular needs at any given time. In some instances, for example, an individual may require a specialist to diagnose and treat less common ailments. In other instances, the individual's health condition may be more common or routine such that a generalist healthcare provider would be well-suited to treat the patient's condition. Complex payment plans and insurance schemes can further increase the difficulty involved in accessing the healthcare system and obtaining appropriate services.

To improve the ability for patients to access healthcare, as well as to provide better consistency, efficiency, and quality of care, many healthcare provider organizations (e.g., clinics and hospitals) operate a system of primary care. Under such a system, patients are each assigned a primary care provider (e.g., a physician or physician's assistant) to take primary responsibility for the patient's care. The primary care provider can provide a consistent point of access for a patient to the healthcare system as a whole. For example, the primary care provider may be a generalist (e.g., a family physician, pediatrician, or internal medicine practitioner) that is trained to deliver a wide range of medical services to a population of patients. Common ailments may be diagnosed and treated by the primary care provider, and the primary care provider may also provide preventative services, perform checkups, and more. If a patient requires treatment from a specialist, the primary care provider may refer patients to the specialist and oversee the patient's progress with a primary care provider. Although it may not always be possible for the patient to see his or her primary care provider, the primary care provider may be the preferred provider for a patient when the patient schedules an appointment at a hospital, clinic, or other healthcare organization.

By consistently interacting with the same primary care provider over an extended time, the patient and primary care provider may develop a relationship of trust and the provider can better know the patient's health conditions and needs. But in addition to improved relationships between the patient and an individual provider, primary care practice also benefits the primary care provider and the healthcare organization associated with the primary care provider. Providers are often more efficient in interacting with their own patients (e.g., patients for whom the provider is the stated primary care provider) because there is a pre-established relationship of trust and a familiarity with the patients' health history and records. Therefore, providers may see more patients and provide better care, resulting in improved patient outcomes and increased revenues.

For example, some provider organizations are transitioning from fee-for-service arrangements to value-based contracts with insurance companies and other payer organizations. Under value-based contracts, the provider organizations typically assume the risk of patients requiring a higher than average cost of healthcare services. In order to maintain profitability under value-based contracts, provider organizations are incented to provide care that leads to the best patient outcomes at the lowest costs. Primary care is one way in which provider organizations strive to achieve efficiency in the delivery of healthcare services. For example, a primary care provider may more reliably perform screening, checkups, and other preventative services for patients that would allow detection of health issues at an earlier, more cost-effective stage.

### SUMMARY

This specification discloses computer-based systems, methods, devices, and other techniques for managing patient panels for primary care providers.

The process of assigning patients to a primary care provider is generally referred to as empanelment. For example, a family physician may have a primary care practice for a panel of patients that identify the physician as their primary care provider. As the primary care provider, the physician should be the primary point of contact for providing primary care to the patients on the physician's panel. Additionally, the size of the physician's panel can be indicative of an overall size of the physician's practice and a volume of primary care services that the physician delivers over a period of time, e.g., on an annual or quarterly basis. To efficiently utilize a given primary care provider in a healthcare provider organization, the organization may prescribe policies or guidelines that specify a target size of a primary care panel assigned to the provider. For example, the organization may set a target panel size between 600 and 800 patients in order to ensure that a full-time provider is efficiently utilized. A panel size under the minimum target may result in an underutilized provider, whereas a panel size that exceeds the maximum target may result in an overutilized provider that is more susceptible to burnout and mistakes.

Patient panels for primary care providers have traditionally been tracked based on designations of primary care providers in electronic medical records of patients of a healthcare organization. For example, an electronic medical records system may include a virtual medical records file for each patient, where the virtual file contains information about the patient's health conditions and interactions (e.g., visits) with one or more providers of the healthcare organization over time. The virtual file for each patient may further include a primary care provider field that holds a value identifying a stated primary care provider for the patient. The stated primary care provider is the primary care provider that is formally assigned to the patient, e.g., based on a manual designation from a system administrator according to the preferences of the primary care provider and/or the patient. For example, when a patient first seeks services from a healthcare provider organization, the patient may be assigned a particular primary care provider from among many possible primary care providers according to established guidelines of the organization. For instance, the patient may express interest in a particular primary care provider, and the organization may oblige by designating the patient's desired provider as the stated primary care provider in the patient's medical records file, whether a physical file or a virtual file or both. On the other hand, if the desired primary care provider already has a full panel or is no longer accepting new patients, the organization may work with the patient to identify another primary care provider for the patient.

In practice, patients do not always see their stated primary care providers to receive primary care services (e.g., due to scheduling incompatibilities between the stated primary care provider, changing preferences of the patient or stated primary care provider, or other factors), and primary care providers sometimes see patients who are not on their stated panels. As a result, the benefits of the primary care system may be at least partially curtailed. It can also be more difficult for providers and healthcare organizations to track patients, understand the actual burdens imposed on providers, and to deliver holistic health services to patients.

The techniques described herein may, in some implementations, aid providers and healthcare organizations in managing primary care patient panels so as to detect and reduce occurrences of patients interacting with non-stated primary care providers, and conversely, to detect and reduce occurrences of primary care providers seeing patients who are not on the providers' stated panels. In some implementations, this is achieved by determining weighted patient panels for primary care providers separately from the providers' stated patient panels, analyzing the stated and weighted patient panels, and adjusting the size and/or composition of the stated panels according to optimization criteria. Whereas the stated patient panel generally results from formal designation of primary care providers for individual patients regardless of which provider(s) the patients are actually interacting with (e.g., visiting) to receive primary care services, the weighted panel for a particular primary care provider represents the panel that the primary care provider has actually interacted with over a recent period of time. For example, if patient A has designated doctor X as her stated primary care provider, but has visited doctor Y four times more than doctor X over the last 36 months for primary care services, then doctor Y may be deemed the weighted primary care provider for patient A (rather than doctor X). Patient A would then appear on doctor Y's weighted panel, but not on doctor X's stated panel. Likewise, patient A would appear on doctor X's stated panel, but not on doctor Y's stated panel.

By determining the weighted patient panel for a primary care provider, the primary care provider can gain greater insight into the patients that he or she is actually seeing. The primary care provider may agree, for example, to assign a particular patient that currently has no stated primary care provider to that provider's stated panel as a result of identifying that the patient is on the provider's weighted panel, especially since the provider is already seeing the patient more than other providers in the system. Likewise, a patient that is on a first primary care provider's weighted panel but on a second primary care provider's stated panel may be identified as a candidate patient to transfer to the first primary care provider's weighted panel (e.g., subject to approval of the first primary care provider, the second primary care provider, or both). As such, the difference between a primary care provider's stated and weighted panels may be narrowed so as to reduce a number of non-matching patients who appear on a provider's weighted panel without appearing on the provider's stated panel.

In some implementations, the techniques disclosed herein utilize patient risk factors to analyze and/or formulate primary care panels. For example, based on patient demographic data (e.g., age and gender), health data, social determinants data, or a combination of these and/or other factors, the system may generate a risk score for a patient that indicates an estimated amount or cost of healthcare services (e.g., primary care services) that the patient is likely to consume over a period of time. Under a value-based contract between a healthcare provider organization and a payer organization, the provider organization may be paid a pre-defined amount to provide healthcare services to treat a patient for a particular condition. The provider organization can realize a profit from the value-based contract by delivering services more efficiently to the patient, but also risks a loss from the contract if services are delivered inefficiently. The risk score for a patient can be a predictive measure of how efficiently services can be delivered to the patient. For example, a patient that suffers from obesity and substance addictions may require more primary care services (and other healthcare services) than other patients of a same demographic category. As such, the risk score for that patient may be relatively high because the patient is expected to more complex and more frequent care over time. Moreover, the complexity of a primary care panel may be measured not just by the number of patients on the panel but also by the anticipated amounts and types of services anticipated to be consumed by patients on the panel, as indicated by their risk scores. As such, the assignment of risk scores to patients on a primary care panel can provide greater insight into characteristics of the panel, which can be then be considered for a range of purposes such as sizing the panel appropriately for a particular primary provider, identifying top performing primary care providers, determining provider compensation, as well as other purposes.

Some implementations of the subject matter disclosed herein include a computer-implemented method, e.g., a method for empaneling primary care providers in a healthcare organization. The method can be performed by a system of one or more computers in one or more locations. The system receives, from a first client, a request for primary care panel information for a particular primary care provider (PCP) in the healthcare organization. The system searches a corpus of patient data that identifies a set of patients of the healthcare organization to determine a first set of patients for which the particular PCP is designated as a stated PCP for the patient. The first set of patients form a stated patient panel for the particular PCP. The system also searches the corpus of patient data to determine a second set of patients for which the particular PCP is designated as a weighted PCP for the patient. For each patient in the second set of patients, the particular PCP is designated as the weighted PCP for the patient based on a quantity of interactions between the patient and the particular PCP during a past time interval with respect to quantities of interactions between the patient and other PCPs during the past time interval. The second set of patients form a weighted patient panel for the particular PCP. The system can generate a response to the request for primary care information for the particular PCP, where the response characterizes information about at least one of the stated patient panel for the particular PCP or the weighted patient panel for the particular PCP. The system can then provide the response to the first client to cause presentation of the information about the at least one of the stated patient panel for the particular PCP or the weighted patient panel for the particular PCP in a graphical interface on a display of the first client.

These and other implementations can optionally include one or more of the following features. The system can determine a first panel score that is based on one or more characteristics of the stated patient panel for the particular PCP. The one or more characteristics of the stated patient panel from which the first panel score is determined can include a size of the stated patient panel. The one or more characteristics of the stated patient panel from which the first panel score is determined can include a risk profile of the stated patient panel, wherein the risk profile indicates an amount of healthcare services that are estimated to be consumed by patients in the stated patient panel. The system can further determine, a second panel score that is based on one or more characteristics of the weighted patent panel for the particular PCP.

The system can identify that a value that is based on the first panel score for the stated patient panel and the second panel score for the weighted patient panel satisfies one or more criteria. In response to identifying that the value that is based on the first panel score for the stated patient panel and the second panel score for the weighted patient panel satisfies the one or more criteria, the system can invoke a process to adjust the stated patient panel for the particular PCP.

The value that is based on the first panel score for the stated patient panel and the second panel score for the weighted patient panel can be a ratio or a difference of the first panel score and the second panel score, wherein the one or more criteria is a threshold ratio or a threshold difference, respectively.

Invoking the process to adjust the stated patient panel for the particular PCP can include prompting the particular PCP to add a patient from the weighted patient panel to the stated patient panel.

A level of compensation for the particular PCP can be determined based at least in part on the first panel score for the stated patient panel.

The system can determine, for each patient in at least a subset of the set of patients of the healthcare organization, a risk score that indicates an amount of healthcare services that are estimated to be consumed by patients in the stated patient panel. Generating the response to the request for primary care information for the particular PCP comprises generating a report that lists patients in at least one of the stated patient panel or the weighted patient panel, wherein the listed patients in the report are sorted based at least in part on the risk scores for the listed patients. Determining the risk score for a given patient in the at least the subset of the set of patients can include determining a hierarchical condition category (HCC) score for the given patient. The risk score for a given patient in the at least the subset of the set of patients can be determined based at least on two or more of demographic information for the given patient, health information for the given patient, and social determinant information for the given patient.

For each patient in the at least the subset of the set of patients of the healthcare organization, the system can identify a set of one or more characteristics for the patient, select, from a set of possible risk models, a particular risk model to apply to the patient, and determine the risk score for the patient using the selected particular risk model.

The set of one or more characteristics for the patient can include at least one of age of the patient or a social determinant of the patient.

The system can further perform operations that include identifying a first patient as a result of the first patient being on the stated patient panel for the particular PCP but not being on the weighted patient panel for the particular PCP; identifying a second PCP for which the first patient is on a weighted patient panel for the second PCP; prompting the second PCP to add the first patient to a stated patient panel for the second PCP; prompting the particular PCP to transfer the first patient to the stated patient panel for the second PCP; and in response to (i) receiving an indication that the second PCP approves adding the first patient to the stated patient panel for the second PCP and (ii) receiving an indication that the particular PCP approves transferring the first patient to the stated patient panel for the second PCP, removing the first patient from the stated patient panel for the particular PCP and adding the first patient to the stated patient panel for the second PCP.

Prompting the second PCP to add the first patient to the stated patient panel can include generating an alert for the second PCP that is accessible via a graphical interface of a patient empanelment dashboard, email, or a Short Message Service (SMS) message.

The system can determine a value that indicates an amount of time that the particular PCP spends interacting with patients on the stated patient panel for the particular PCP. In some implementations, the system can determine a second value that indicates a total amount of time allotted to the particular PCP for interacting with patients, and can determine a third value that indicates a ratio of the value that indicates the amount of time that the particular PCP spends interacting with patients on the stated patient panel for the particular PCP and the second value that indicates a total amount of time allotted to the particular PCP for interacting with patients.

The first client can be a computing device that communicates with the system over a network. In other implementations, the first client is included among the one or more computers of the system.

Generating the response to the request for primary care information for the particular PCP can include formatting an electronic document that includes the information about at least one of the stated patient panel for the particular PCP or the weighted patient panel for the particular PCP. Providing the response to the first client can include transmitting the formatted electronic document to the first client.

Generating the response to the request for primary care information for the particular PCP can include generating an extensible markup language (XML) document that includes the information about at least one of the stated patient panel for the particular PCP or the weighted patient panel for the particular PCP. The first client can be configured to format a second document for presentation in the graphical interface using the information from the XML document.

Some implementations of the subject matter disclosed herein include one or more computer-readable media having instructions stored thereon that, when executed by one or more processors, cause the one or more processors to perform operations from any of the computer-implemented methods or other techniques disclosed herein. The computer-readable media can include non-transitory media, such as a hard disk, a solid state memory storage device, an optical disk, or other media that can be encoded with machine-readable instructions. Some implementations of the subject matter disclosed herein include a computing system that includes one or more processors and one or more computer-readable media having instructions stored thereon that, when executed by the one or more processors, cause the one or more processors to perform the operations disclosed herein.

Various features and advantages of the techniques disclosed herein are described in the detailed description and would be understood by persons of ordinary skill in the field in view of the entire specification.

### DESCRIPTION OF DRAWINGS

FIG. 1 depicts an environment of an example patient management computing system that maintains, analyzes, and provides information about patient panels for primary care providers.

FIG. 2 shows a Venn diagram illustrating the relationship of a stated patient panel to a weighted patient panel.

FIG. 3 depicts a flowchart of an example process for assessing a level of risk for a patient in a primary care system.

FIG. 4 is a flowchart of an example process for selecting a weighted primary care provider for a patient.

FIG. 5 is a flowchart of an example process for comparing and analyzing patient panels for primary care providers.

FIG. 6 is a flowchart of an example process for re-assigning patients on stated patient panels between two or more primary care providers.

FIG. 7 shows a table for presentation in a graphical interface of a patient panel management system that summarizes the stated and weighted panels for a particular primary care provider.

FIG. 8 shows a table for presentation in a graphical interface of a patient panel management system that identifies patients in a primary care provider's stated match and unweighted panel.

FIG. 9 shows a table for presentation in a graphical interface of a patient panel management system that identifies patients in a primary care provider's weighted panel that do not have a stated primary provider.

FIG. 10 shows a table for presentation in a graphical interface of a patient panel management system that identifies patients in a primary care provider's stated panel that are assigned to a different provider's weighted panel.

FIG. 11 shows a plot of the distribution of patients on a primary care provider's stated and weighted patient panels, organized by age and risk categorization (e.g., HCC risk priority).

FIG. 12 shows a table for presentation in a graphical interface of a patient panel management system that shows the distribution of patients in a primary care provider's panel by age range and risk category.

FIG. 13 shows a table for presentation in the graphical interface of a patient panel management system that shows a risk adjusted panel of a primary care provider.

FIG. 14 shows a table for presentation in the graphical interface of the patient panel management system that shows all patients in a primary care provider's stated panel.

FIG. 15 shows an example of a computing device and a mobile computing device that can be used to implement the techniques described herein.

### DETAILED DESCRIPTION

This specification generally discloses computer-based systems, methods, devices, and other techniques for managing patient panels for primary care providers. In some implementations, a computing system can be configured to analyze patient data for a large number of patients and primary care providers in a provider organization such as a hospital or clinic. The computing system may employ various techniques to efficiently analyze the patient data and determine patient panel information for presentation to the primary care providers, hospital administration, or other interested parties who are permitted access to such information under applicable laws and regulations. Further detail concerning these and other techniques are depicted in and discussed in reference to the drawings.

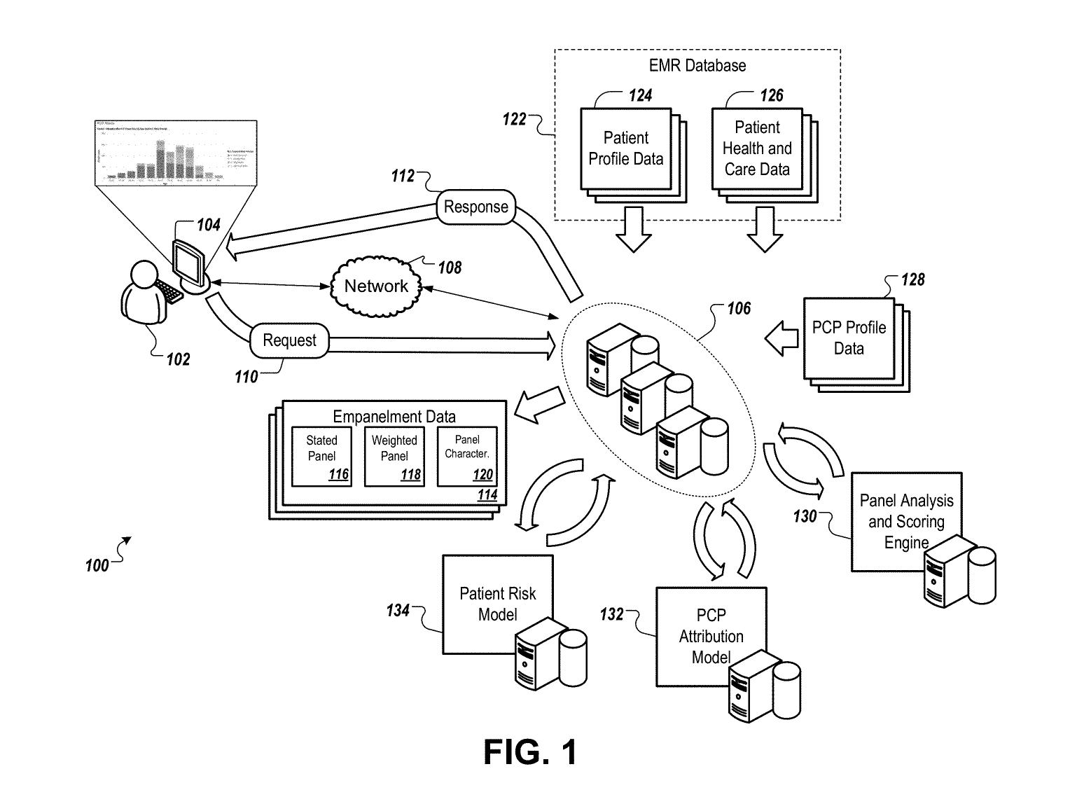

FIG. 1 depicts an environment 100 of an example patient management computing system 108 that maintains, analyzes, and provides information about patient panels for primary care providers. The computing system 106 may be implemented as one or more computers in one or more locations. In some implementations, the system 106 may be implemented as a single server or a bank of servers locally maintained by a healthcare provider organization. In other implementations, the system 106 may be implemented as one or more servers located remotely from the provider organization. For example, a healthcare provider organization may contract with a software development organization that operates a cloud-based patient management platform on system 106. Agents of the provider organization (e.g., user 102) may then access the cloud-based platform through client devices, e.g., client device 104, over a network 108 such as the Internet or a local area network. For example, a user 102 of the client device 104 may request patient panel information for one or more primary care providers over network 108. The computing system 106 may process the request 110 and return a response 112, e.g., a communication that identifies patient panels for the particular primary care provider(s) specified in the request. The client device 104 may be any suitable computing device that is part of or capable of communication with the computing system 106, e.g., a desktop computer, a notebook computer, a smartphone, a tablet computing device, a “smart” watch, or other forms of wearable or mobile computing devices.

In general, the computing system 106 is configured to process various information about patients and primary care providers to assign patient panels to primary care providers, to perform analysis on the patient panels, generate reports and other presentable information about the patient panels to interested and permissible users, and to provide an interface in which modifications can be efficiently made to a patient panel. The computing system 106 can provide users with greater awareness into the demands on primary care providers by determining and analyzing two distinct types of patient panels, i.e., stated patient panels 116 and weighted patient panels 118. A stated patient panel 116 for a particular primary care provider refers to the set of all patients who are formally assigned to the primary care provider. For example, a physician listed in a patient's medical records file as the primary care provider for that patient is referred to as the stated or designated primary care provider. All such patients whose medical records files list the same primary care provider collectively form the stated patient panel for that primary care provider.

In addition to stated patient panels 116, the computing system 106 is also configured to determine and maintain information about weighted patient panels 118. The weighted patient panel 118 for a particular primary care provider is not based on formal designations of the primary care provider to a group of patients, but instead identifies a group of patients who the primary care provider actually tends to see over a period of time. For example, a patient that has received primary care services from a first primary care provider more frequently over a period of time than any other known primary care provider may appear on the first primary care provider's weighted patient panel 118. The patient may be assigned to the first primary care provider's weighted panel 118 regardless of who is formally stated as that patient's primary care provider (or even whether the patient a stated primary care provider at all). In many instances, the same patient may appear on both the stated patient panel 116 and the weighted patient panel 118 for a primary care provider. In other instances, however, a patient may occur on either, but not both, the stated panel 116 and the weighted panel 118. These latter instances may occur, for example, when a patient tends to see a different primary care provider from the provider stated in the patient's medical records file or for patients who have not identified a stated primary care provider.

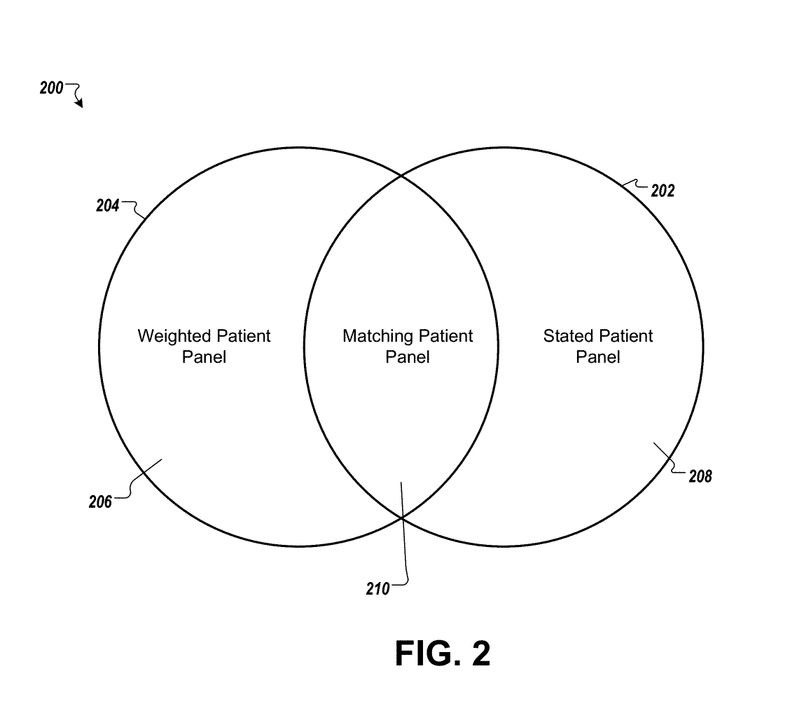

To illustrate the concept of stated and weighed patient panels, refer to the Venn diagram 200 depicted in FIG. 2. The diagram 200 represents an example relationship between a stated patient panel 202 and a weighted patient panel 204 for a primary care provider. The stated patient panel 202 includes two sub-panels of patients: a non-matching stated panel 208 and a matching panel 210. Similarly, the weighted patient panel 204 includes a non-matching stated panel 208 and the matching patient panel 210. The matching panel 210 consists of patients that appear on the same primary care provider's stated and weighted panels. For effectively managed and implemented panels, the relative size of the matching panel 210 would be expected to increase as a result of the primary care provider tending to primarily interact with (e.g., deliver primary care services to) patients on the provider's stated panel. The greater difference that exists between the non-matching panels 206, 208 and the matching panel 210 indicates a wider separation between the formal designation of the primary care provider and the reality of who the patients are actually seeing for their primary care needs.

Referring back to FIG. 1, the computing system 106 can process various types of data to determine and analyze patient panel information. First, the system 106 may obtain patient information from an electronic medical records database 122. The database 122 may be of any suitable type such as a relational database, navigational database, or a post-relational database. As used herein, a database refers to an organized collection of data stored in a machine-readable storage device, although the manner of organization may vary in different implementations. The electronic medical records database 122 may include a set of electronic medical records files (virtual medical records files) for a set of patients, e.g., a set of patients of a hospital, clinic, or other healthcare provider organization.

In some implementations, the medical records file for a given patient represented in the database 122 includes patient profile data 124, patient health and care data 126, or both. The patient profile data 124 can include a collection of data representing various attributes of a patient. For example, the patient profile data 124 may include data indicating the name of a patient, a global identifier assigned to the patient, demographic information for the patient (e.g., age/date of birth and gender), a residence address or other geographical identifier for the patient, and a stated primary care provider that has been assigned to the patient. The stated primary care provider may be assigned, for example, based on which provider the patient initially visited when the virtual file for the patient was created, or it may be updated at later times if the user or a provider indicates an intent to change the stated primary care provider. In some implementations, the patient profile data 124 further includes social determinants data that identifies social determinants applicable to the patient. The World Health Organization states that “[t]he social determinants of health are the conditions in which people are born, grow, live, work, and age,” which can be “shaped by the distribution of money, power and resources at global, national and local levels.” (WHO|Social Determinants of Health, http://www.who.int/social determinants/sdh definition/en/). The social determinants data stored in EMR database 122 can identify one or more such social determinants for a patient, such as a living condition (e.g., whether the patient is homeless or has a regular residence), an employment condition (e.g., whether the patient has stable employment), a wealth condition (e.g., whether the patient's wealth or income is within a threshold amount of the federal poverty line), or other social factors of the patient for which a link has been shown to potentially impact the patient's health. In some implementations, information in the patient profile data 124 is voluntarily shared by patients via responses to questions on an electronic or paper-based form. In some implementations, the database 122 is encrypted and data stored in the database 122 is restricted from general access to users under applicable laws and regulations, such as the Health Insurance Portability and Accountability Act of 1996 (HIPAA).

The electronic medical records for patients represented in the database 122 can also include patient health and care data 126. The patient health and care data 126 represents information about the medical history of a patient such as past health conditions of the patient (e.g., results of physicals, colonoscopies, previous diagnoses), current health conditions of the patient (e.g., the result of a recent physical), and visitation data that indicates each interaction of the patient with one or more primary care providers and, optionally, one or more other healthcare providers over a period of time. The visitation data can contain, for example, for each visit to a healthcare provider over a period of time, data that indicates a date of the visit, an indication of the primary care provider that attended the visit (i.e., who the patient interacted with to receive primary care or other healthcare services), and any results of the visit such as a diagnosis, health condition, prescriptions or referrals made by the primary care provider, and any other notes or memos that the primary care provider made to file.

The computing system 106 is also configured to access a database storing primary care provider profile data 128. In some implementations, the primary care provider profile data 128 is stored in a same database as the electronic patient medical records, e.g., EMR database 122. In other implementations, the primary care provider profile data 128 is stored separately from the electronic patient medical records. In general, primary care profile data 128 includes profile information for one or more primary care providers, e.g., all or some of the primary care providers in a healthcare organization. For each primary care provider represented in the dataset 128, the profile information can include such data as to indicate a name of the primary care provider, a global unique identifier for the primary care provider, a type or specialization of the primary care provider (e.g., physician, physician's assistant, resident, pediatrician, family physician, internal medical physician), employment status (e.g., full-time employee vs. part-time employee), and other information usable by the system 106 to analyze and determine patient panels for the primary care provider. In some implementations, the primary care provider profile data 128 can further include patient visitation or interaction data that includes records of services performed for patients or other interactions of the primary care provider with patients over a period of time. In other implementations, such data is maintained exclusively in the electronic medical records database 122, although the data may be linked by reference between the patient health and care data 126 and the primary care provider profile data 128.

The environment 100 can further include a panel analysis and scoring engine 130, a primary care attribution model 132, and a patient risk model 134. Each of these components 130, 132, and 134 may be implemented as computer programs on one or more computers, whether together or separately from each other. In some implementations, as illustrated in FIG. 1, components 130, 132, and 134 are separate from the patient management computing system 106, although the system 106 is configured to interact with each of the components 130, 132, and 134. For example, each component 130, 132, and 134 may expose an application programming interface (“API”) to the computing system 106 that allows the system 106 to call particular functions or services implemented by the respective components and obtain a response. In other implementations, one, two, or all of the components 130, 132, and 134 may be implemented as part of the computing system 106.

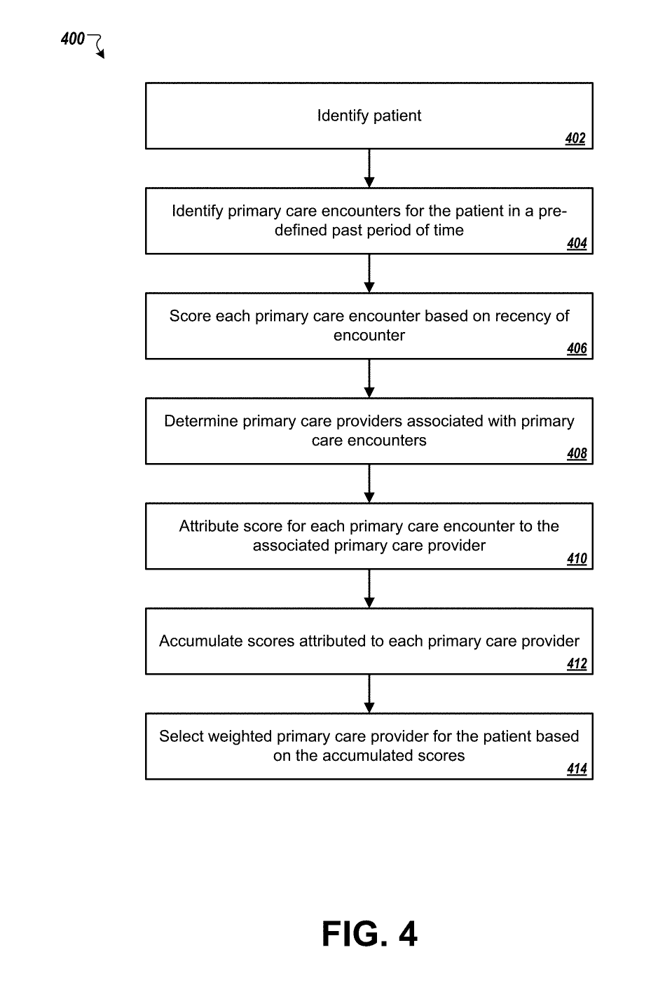

The primary care provider attribution model 132 is configured to process records of provider-patient interactions to determine a weighted primary care provider to assign to a patient, or conversely to determine a patient to include on a primary care provider's weighted panel 118. For example, the system 106 may obtain patient visitation or interaction data from the electronic medical records database 122, and may analyze the data with the PCP attribution model 132. In some implementations, the model 132 is configured to assign a patient to a primary care provider's weighted panel 118 based on a weighted accumulation of interactions (encounters) between the patient and the primary care provider over time. For example, each appointment that the patient has with a primary care provider from 24-36 months in the past may be assigned 1 point; each appointment that the patient has with a primary care provider from 12-24 months in the past may be assigned 2 points (thus weighting more recent interactions greater than more distant interactions); and each appointment that the patient has with a primary care provider from 0-12 months in the past may be assigned 3 points. The points for each primary care provider seen by the patient over the past 36 months may then be totaled and the provider with the highest point total is designated the weighted primary care provider for the patient. In some implementations, the attribution model 132 uses other point allocations and decay functions that express how points are diminished over time. The process of attributing a weighted primary care provider to a patient is discussed in further detail with respect to FIG. 4.

The patient risk model 134 is configured to quantify a level of risk associated with patients represented in the EMR database 122. In some implementations, the level of risk quantified by the risk model 134 is an estimate of the cost of healthcare services that will be consumed by a patient. The risk score can reflect, for example, a level of risk that the cost of healthcare services that are anticipated for the patient will exceed the payments that are anticipated to be received for providing such services to the patient. This level of risk can be expressed as a risk score, where the risk score is a value on a defined scale that corresponds to a level of risk associated with the patient.

Although direct predictions of the cost of healthcare services that are anticipated for a patient and predictions of the payments that would be received for the patient may be difficult to determine, the system can, in some implementations, process other more readily available information in determining the risk score for a patient so as to estimate the financial risk to the healthcare provider or provider organization. For example, in some implementations, the risk score assigned to a patient is a health condition category (HCC) score that is based on demographic information (e.g., age and gender) and a health status of the patient. Health status may reflect acute and/or chronic conditions of the patient as indicated by the patient health and care data 126. In some implementations, the risk score is further determined based on the social determinants of health for the patient, as indicated by the patient profile data 124. For example, an adult that has achieved at least a high school level of education may be deemed a lower risk than adults that have not attained a high school diploma. Accordingly, the risk score for adults that have not achieved a threshold level of education may reflect a higher risk than if the adults had achieved the threshold level of education. In some implementations, social determinants and other risk factors are only implemented in the risk model 134 if the factors have been empirically determined as statistically significant contributors to the health status of patients, or to the level of primary care services, or other healthcare services, provided to the patients.

In some implementations, the patient risk model system 134 is configured to select particular risk factors and/or a particular one of a set of candidate risk models based on one or more specified criteria. For example, the system 134 may weigh certain health conditions of a patient differently in the risk score calculation depending on the age or age range of the patient. Social determinants or conditions such as autism, Down's syndrome, obesity, mental health conditions, that are applicable to a patient may also influence the selection of risk factors and/or a candidate risk model to apply in determining a risk score for a patient.

The panel analysis and scoring engine 130 is configured to analyze a patient panel, e.g., a stated patient panel 116 or a weighted patient panel 118, and to determine values for one or more metrics of the panel. In some implementations, the panel analysis and scoring engine 130 can also compare panels, such as the stated panel 116 and weighted panel 118 for a particular primary care provider, or can perform inter-provider comparisons of panels for multiple primary care providers. The values of the metrics determined by the panel analysis and scoring engine 130 can be applied to various ends such as evaluating the composition of a primary care provider's panel, predicting the complexity or workload associated with a panel, or determining compensation for a primary care provider. For example, in some implementations, the analysis and scoring engine 130 can determine a value for a complexity metric of a patient panel. The complexity metric is a measure of the complexity of a patient panel, both in terms of the number of patients in the panel and the complexity of the patients in the panel. Conventionally, patient panels have been assessed in terms of their size (e.g., the number of patients in the panel), but a purely size-based assessment does not capture patient characteristics that can significantly impact the burden of the panel to a primary care provider. Therefore, the panel analysis and scoring engine 130 accounts for patient characteristics in addition to panel size in the panel complexity metric. In some implementations, the scoring engine 130 determines a value for the panel complexity metric using patient risk scores as indicated by the patient risk model system 134. For example, the scoring engine 130 may adjust the value of the complexity metric for a given size of a panel to represent higher complexity if the panel composition is skewed toward higher risk patients. Conversely, the scoring engine 130 may adjust the value of the complexity metric for a given size of a panel to represent lower complexity if the panel composition is skewed toward lower risk patients.

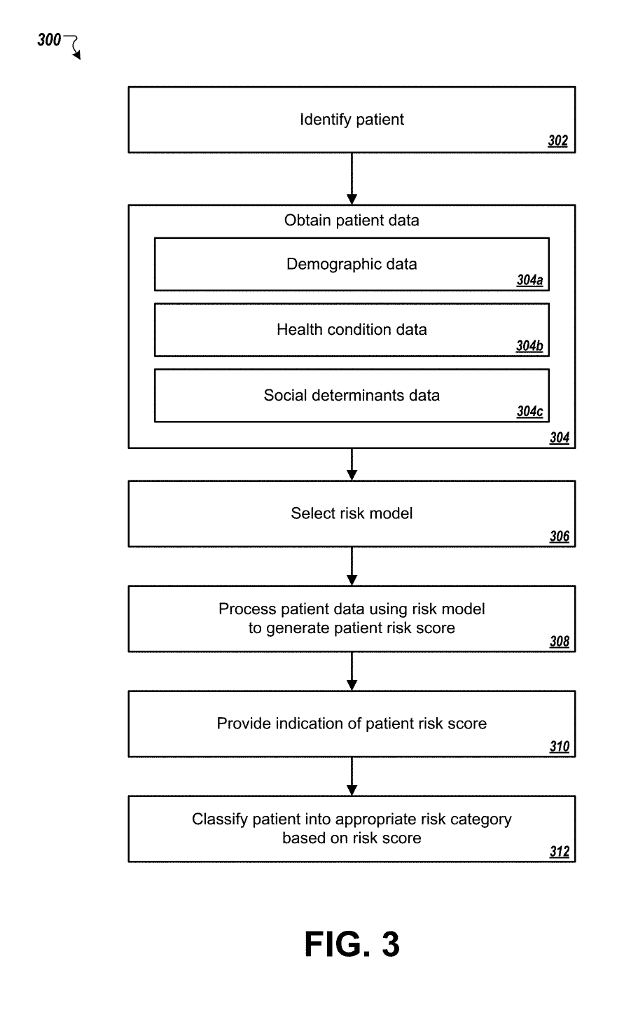

Turning to FIG. 3, a flowchart is shown of an example process 300 for assessing a level of risk for a patient in a primary care system. In some implementations, the process 300 is performed by a computing system that implements a patient risk model, e.g., the patient management computing system 106 and/or patient risk model system 134. The process 300 can determine a risk score for a patient that indicates an estimated cost of care for the patient. Higher costs of care for a patient can potentially represent higher risk for healthcare providers, particularly under the terms of value-based contracts in which payers do not pay per service performed but rather pay a lump sum for an entire course of treatment or services for a given condition of a patient. For the purpose of panel management, risk scores are also indicative of the relative complexity of a patient. An older and sicker population of patients tend to require more care than younger or healthier populations, and thus the composition of patients in a panel can result in significantly different workloads or challenges for a primary care provider.

At stage 302, the system identifies a target patient, i.e., the patient for which a risk assessment is to be performed. At stage 304, the system obtains patient data that is indicative of the level of risk for a patient. In some implementations, the patient data includes one or more of demographic data 304a, health and care data 304b, and social determinants data 304c. In some implementations, the patient data is pre-stored in an electronic medical records database such as EMR database 122. The system can query the database to obtain any of the data 304a, 304b, or 304c. The database may be encrypted to protect patient privacy and confidentiality, in which case the system may provide credentials that the database management system can process to verify that the requestor is an authorized user. The demographic data 304a can include information such as the age and/or gender of the patient. The health and care data 304b can include information such as current or past medical conditions of the patient, whether acute or chronic and records previous interactions with primary care and/or other healthcare providers. The social determinants data 304c indicates information about the social determinants of health of the patient, such as living condition, socioeconomic status, and/or a highest level of attained education.

At stage 306, optionally, the system selects a particular risk model to apply for the patient's risk assessment. In some implementations, the system maintains multiple candidate risk models. Each risk model may be associated with different sets of characteristics or attributes of a patient. For example, patients that have conditions such as autism or Down's syndrome may be scored using certain risk factors that are not otherwise applicable to patients that do not have these conditions. A risk model that accounts for the risk factors associated with autism or Down's syndrome may thus be selected if the target patient has one of these conditions. Patients may be categorized in other manners as well that dictate selection of different risk models. A pediatric patient, for instance, may require a different risk model than an adult patient. In other implementations, a common risk model can be applied for all patients.

At stage 308, the system processes the patient data that was obtained at stage 304 using the selected risk model to generate a risk score for the target patient. For example, a health condition category (HCC) risk score may be determined. The selected model may alternatively determine a modified HCC score to account for risk factors (e.g., social determinants of health) that are not traditionally considered in assessing risk of a patient.

At stage 310, the system outputs an indication of the determined risk score for the patient. For example, the risk score may be presented in a report to a primary care provider or a healthcare administrator. In some implementations, the score is cached or otherwise stored in memory for subsequent use in determining or analyzing patient panels. Because a healthcare organization may have many primary care patients (e.g., hundreds, thousands, or even millions of patients), the process of determining current risk scores for the patients may be a time-consuming process, and one that is infeasible without the use of a computing system. A patient panel management system may schedule a periodic job, for example (e.g., on a daily, weekly, or monthly basis), to determine updated risk scores and other information relevant to patient empanelment. The system may process vast quantities of data for a large patient population in a timely manner. Later, when the system is called to determine a patient panel for a primary care provider, current patient risk scores and other relevant data are already stored and available to the system, thereby allowing panel management tasks to be performed quickly and with minimal latency to the user.

In some implementations, the system (e.g., patient risk model system 134 and/or patient management computing system 106) is configured to classify patients into different risk categories based on their risk scores. A risk category can represent a broad range of patients having similar risk scores. For example, HCC risk scores for patients are typically in the range from just above 0 to about 12 or 13, where higher numbers represent increased risk. The HCC score is often more granular than is necessary to assess the prospective impact of assigning a patient to a primary care provider's panel. Therefore, for example, patients with a lower range of risk scores may be grouped into a low-risk category (e.g., scores from just above 0 to 3); patients with a medium range of risk scores may be grouped into a medium-risk category (e.g., scores from 3 to 7); patients with a high range of risk scores may be grouped into a high-risk category (e.g., scores from 7-11); and patients with a very high range of risk sores may be grouped into a very high risk category (e.g., scores from 11+). The system may then present the risk category of a patient in panel reports to primary care providers, rather than or in addition to the raw risk scores. In some implementations, the reports are filtered to display patients in a panel with only specified risk categories, or the reports are sorted, e.g., to display the highest risk patients toward the top of a list patients in a panel and the lowest risk patients toward the bottom of the list.

FIG. 4 is a flowchart of an example process 400 for selecting a weighted primary care provider for a patient. As discussed with respect to FIG. 1, a patient's weighted primary care provider is the provider that the patient has actually seen most frequently during a recent period of time. The weighted primary care provider for a patient may be, or may not be, the same as the formally stated primary care provider for the patient. In some implementations, the process 400 is performed by a patient management computing system in cooperation with a primary care provider attribution model, e.g., attribution model 132.

The process 400 can begin at stage 402, where the system identifies a target patient, i.e., the patient for which a weighted primary care provider is to be determined. The system accesses the patient's virtual electronic medical records file and identifies each encounter (e.g., each visit, appointment, or interaction) of the patient with a primary care provider in a past period of time (stage 404). The period of time may be set according to a default or user-specified policy. In some implementations, the period of time is 1, 2, or 3 years, or is in the range 3-60 months, and is preferably in the range 12-36 months. The system attributes a score for each encounter to the primary care provider involved in the encounter (stage 406). For example, if the patient obtained primary care services from three different providers in the last six months, each of the three providers would be attributed scores for their encounters with the patients (stage 408). In some implementations, the system applies a weighting function that weights the score attributed to the primary care provider for a given encounter based on how recently the encounter occurred. Encounters that occurred three years ago, although within the window of time identified at stage 404, may be attributed less weight than more recent encounters in the last twelve months, for example. At stage 410, the score assigned to each encounter is attributed to the corresponding primary care provider for the encounter. For each primary care provider that attended to the patient in the recent period of time, the scores attributed to the provider are summed or otherwise combined to generate an accumulated score for the provider. At stage 414, the system then selects a weighted primary care provider for the patient based on the accumulated scores. In some implementations, the provider having the highest accumulated score for the patient is selected as the weighted provider for that patient, although other criteria are possible as well. In the event of a tie, such as two providers that have equally high accumulated scores, only one of the providers is selected as the weighted provider for the patient, while others are not. The selection of the weighted provider in a tie scenario can be based on one or more tiebreaking rules. For example, the system may break a tie by selecting the provider that most recently saw the patient as the weighted provider for the patient.

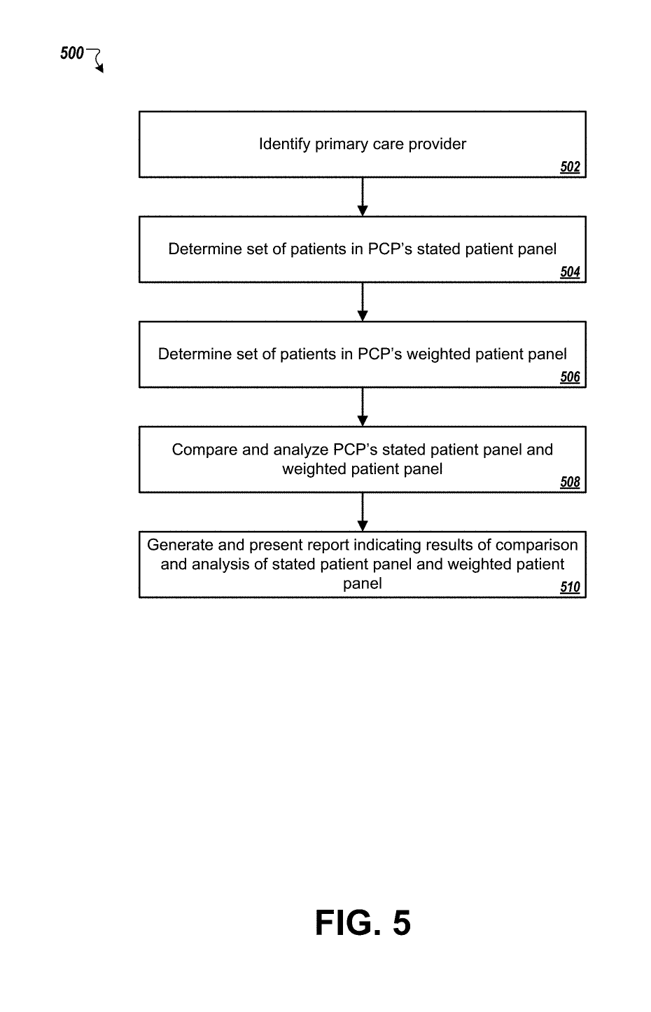

FIG. 5 is a flowchart of an example process 500 for comparing and analyzing patient panels for primary care providers. In some implementations, the process 500 is carried out by a patient management computing system, e.g., system 106.

The process 500 can be performed with respect to a particular primary care provider. The system may identify the primary care provider based on a specification of the provider in a user interface, a query, or other manner. In some implementations, the system selects the provider who is currently logged into the patient management system as the target provider for the process 500 by default. The system determines a set of patients in the primary care provider's stated patient panel (stage 504) and weighted patient panel (506).

At stage 508, the system may compare and analyze the primary care provider's stated and weighted patient panels. For example, the system may determine, for each panel, a number of patients in the panel, a number of matching patients in the panel (i.e., patients that appear in both the stated and weighted panels), a number of non-matching patients in the panels, and/or a percentage of matching or non-matching patients. In some implementations, the system may also compare metrics of the provider's panels to other providers in a health care organization. For example, the system may determine an average (e.g., mean or median) size of a patient panel among a group of primary care providers and may present a table or chart juxtaposing the particular provider's panel size with the average panel size of the group. In some implementations, the system determines a complexity score for the patient's stated and/or weighted patient panel, and may compare the complexity score to the scores of other providers. The results of the analysis can be presented in a graphical interface, e.g., in a dashboard displayed at a client of the primary care provider (stage 510).

In some instances, the system generates a report for presentation to a primary care provider including information about the provider's stated and weighted patient panels. The report can include, for example, a listing of patients in each panel, selected attributes of each patient in the panel, and an electronic link to each patient's file in the electronic medical records database. Upon reviewing the report, a primary care provider or other user (e.g., a clinic administrator) may wish to make changes to the provider's stated patient panel. For instance, if there is a wide divergence between the provider's stated and weighted panels, the system may invoke a process to transfer patients on the provider's non-matching weighted panel to the provider's stated panel. Likewise, patients on the provider's stated panel may be removed, e.g., by transferring selected patients to another provider's stated panel or by deactivating certain patients for which there is no indication of having received primary care services within a threshold period of time (e.g., 4, 5, or 6 years).

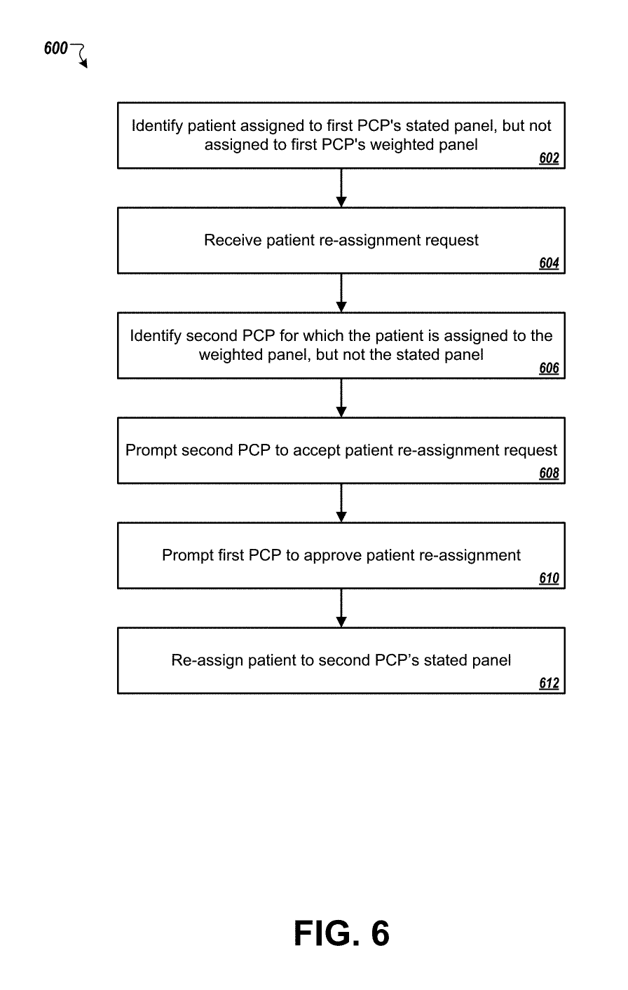

To facilitate the ability to adjust a provider's stated patient panel, the system may perform a process like that depicted in FIG. 6. FIG. 6 is a flowchart of an example process 600 for re-assigning patients on stated patient panels between two or more primary care providers. In general, the process 600 is performed such that the current primary provider (i.e., the provider for whom a patient is to be transferred off his or her stated patient panel), the target primary provider (i.e., the provider for whom the patient is to be transferred onto his or her stated patent panel), or both, can provide an indication of consent to the patient transfer before the transfer is completed. For example, the system may restrict a patient from being moved on or off a primary care provider's patient panel without approval from the primary care provider, or at least without providing the primary care provider with an opportunity to accept or decline a proposed move. In some implementations, a system administrator having overriding privileges that allow patients to be moved among panels without consent of the primary care providers, even if the providers are unable to make non-notified or non-approved transfers themselves.

At stage 602, the system identifies a patient that is a candidate for being transferred from a first primary care provider's stated panel to a second primary care provider's stated panel. The patient may be identified as a candidate as result of being among the first primary care provider's non-matching stated panel. That is, the patient is formally declared on the first primary care provider's stated panel, but has not been actively seen by the first primary care provide and is thus not on the first primary care provider's weighted panel. In some implementations, the system can generate suggestions for the first primary care provider, which are presented in a graphical user interface or otherwise communicated to the first provider. The suggestions may indicate candidate patients to move on or off of the first provider's stated panel based on the candidate patients being in a non-matching group. In some implementations, the system may format a report or a user interface displayed on a display device of the system to highlight the candidate patients. For instance, patients that are candidates to move off the stated panel may be highlighted in one color, and patients that are candidates to move from the non-matching weighted panel to the stated panel may be highlighted in another color. In other implementations, the patient may be manually identified by the first primary care provider based on user input to the system from the first provider. The patient may alternatively be manually identified by the second primary care provider based on the patient being on the second provider's weighted panel.

At stage 604, the system receives a request to re-assign the candidate patient. Because the candidate patient is already on the first primary care provider's stated panel, the system may infer (if it is not explicitly indicated in the request) that the request is to transfer the candidate patient off the first provider's stated panel. In some implementations, the request can be submitted based on user input from the first primary care provider indicating a desire to transfer the candidate patient. In some implementations, the request can be submitted based on user input from the second primary care provider indicating a desire to transfer the candidate patient to the second provider's stated panel.

At stage 606, the system identifies the second primary care provider as the target of the re-assignment request. If the second primary care provider is not the party that initiated the request, the system may automatically determine the second primary care provider as being the target of the re-assignment request based on the second primary care provider having the candidate patient on his or her weighted panel. If other implementations, the party that initiated the re-assignment request may manually specify the target provider.

At stage 608, the system prompts the second primary care provider to accept the patient re-assignment request. In some implementations, the system generates the prompt in a graphical user interface of the patient management system on a display of a client device accessed by the second primary care provider. The prompt may be provided in the form of a notification that is presented directly in, or that is indirectly accessible through, a patient panel management dashboard of the second primary care provider. In some implementations, the system may store data representing addresses of external accounts of primary care providers. Using the addresses, the system may send re-assignment requests and notifications to the external accounts in addition to transmitting the requests and notifications within the patient panel management system. For example, the system may email or transmit a text message (e.g., a Short Message Service message) to the second primary care provider that includes the prompt to accept a patient re-assignment request. In some implementations, the message may contain electronic links or other interface elements that, when selected by the second primary care provider, submits a response to the patient management system indicating the second provider's decision to accept or reject the re-assignment request. In some implementations, the second provider may attach a note along with the response to the first provider, e.g., to permit the second provider to ask questions of the first primary care provider or explain a decision to accept or reject the re-assignment request.

At stage 610, the system may prompt the first primary care provider to approve the patient re-assignment request in a similar manner to how the system prompted the second primary care provider to approve the patient re-assignment request. For example, a prompt may be presented to the first primary care provider in a dashboard interface of the patient management computing system for the first primary care provider. In some implementations, the prompt may also be transmitted to the first primary care provider through one or more external channels such as email, text, mobile applications, or telephone calls. In some implementations, the first provider need not re-approve a re-assignment request if the first provider was the party that invoked the request. Likewise, if the second provider was the party that invoked a re-assignment request, then only the counter-party to the request may be prompted to accept or deny the request.

At stage 612, the system determines whether the required parties have approved the request to re-assign the patient from the first primary care provider's stated panel to the second primary care provider's stated panel. If the request is approved, the system re-assigns the patient accordingly. For example, the system may access the patient's electronic medical records file and re-populate a field that indicates the stated primary provider for the patient with an identifier of the second primary care provider in place of an identifier for the first primary care provider. If the request was not approved, the system blocks the re-assignment until the request is either approved at a later time by the primary care providers or is forced through by a user with higher-level system privileges.

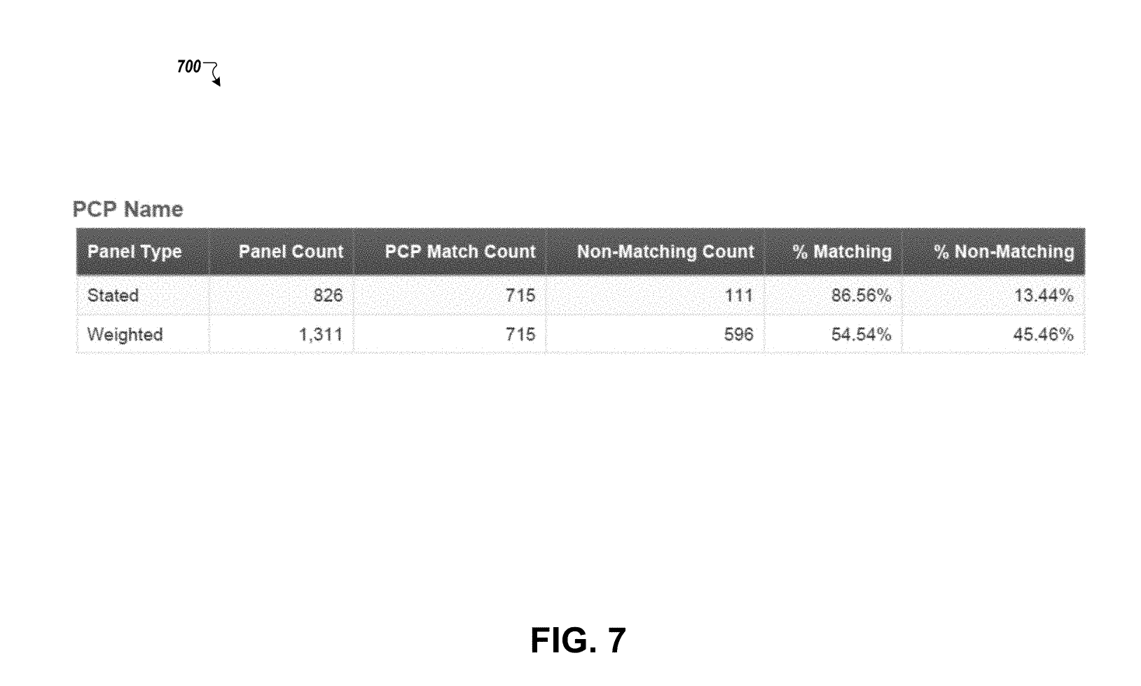

FIGS. 7-14 show screenshots of various aspects of a user interface of a patient panel management computing system. To facilitate users' review and analysis of primary care patient panels, the system may generate a user interface (e.g., a dashboard) that is configured to be presented on the displays of one or more computing devices of the system itself, or of clients that interact with the system. For example, a primary care provider may access the user interface on a personal computing device (e.g., a smartphone, tablet, notebook or desktop computer) via a web-based application or an application installed on the device. The user interface may include various controls, represented as text or other graphical elements in the user interface, with which a primary care provider or other user can interact to access different reports or modules made available through the dashboard, to review and accept or deny patient re-assignment requests, or to propose patient re-assignments based on a review of the provider's stated and weighed patient panels. In some implementations, the system may generate pre-formatted electronic documents containing reports or other panel information. The pre-formatted document may then be transmitted as one or more files to a client device that can be executed and displayed at the client. In other implementations, a structured dataset (e.g., XML) may be transmitted to a client device. An application at the device may then format a document or populate fields in a local form, for example, to display the patient panel information.

FIG. 7 shows a table 700 for presentation in a graphical interface of a patient panel management system that summarizes the stated and weighted panels for a particular primary care provider. The table can include data indicating, for each of the stated and weighted panels, a count of a number of patients in the panel, a number of matching patients that occur in the stated and weighted panels, a number of non- matching patients in the panel (e.g., number of patients that are only in the stated panel but not the weighted panel and vice versa), an indication of the percentage of matching patients in each panel, and an indication of the percentage of non-matching patients in each panel.

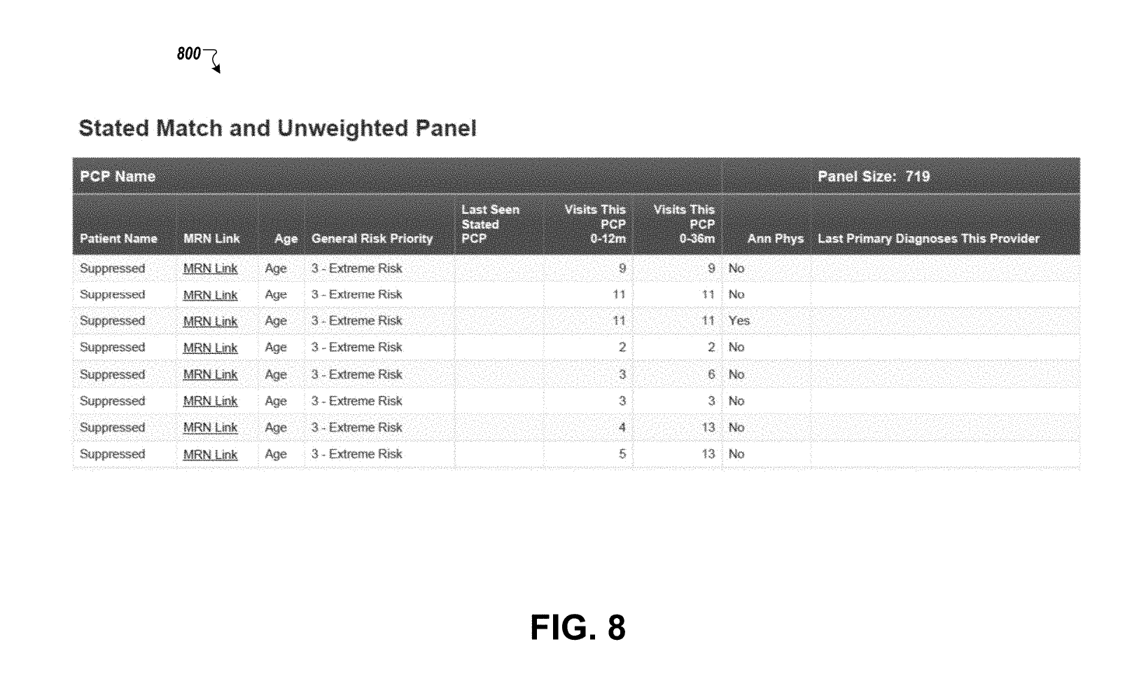

FIG. 8 shows a table 800 for presentation in a graphical interface of a patient panel management system that identifies patients in a primary care provider's stated match and unweighted panel. The table 800 includes a row for each patient, where each row includes a field indicating the patient's name, a link to the electronic medical records file for the patient (MRN Link), an age indicator for the patient, the risk categorization for the patient (General Risk Priority), a date that the patient last saw the stated primary care provider, a number of visits to this primary care provider in the last 12 months and 36 months, whether the patient has had an annual physical in the last year, and the last primary diagnoses from the primary care provider. Of course, different combinations of fields may be displayed for each patient row by default or based upon a user's reconfiguration of the table 800.

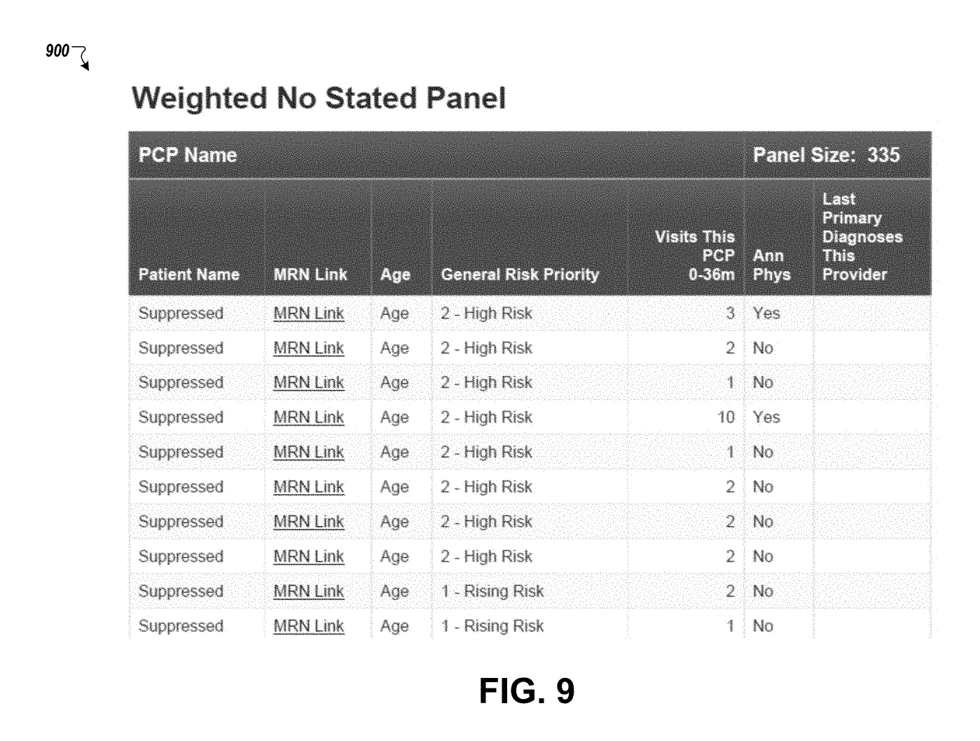

FIG. 9 shows a table 900 for presentation in a graphical interface of a patient panel management system that identifies patients in a primary care provider's weighted panel that do not have a stated primary provider. The weighted primary care provider may review this table 900, for example, to determine whether to add the patients to his or her stated panel, since no re-assignment would be required. The table 900 includes a row for each patient, where each row includes a field indicating the patient's name, a link to the electronic medical records file for the patient (MRN Link), an age indicator for the patient, the risk categorization for the patient (General Risk Priority), a date that the patient last saw the stated primary care provider, a number of visits to this primary care provider in the last 12 months and 36 months, whether the patient has had an annual physical in the last year, and the last primary diagnoses from the primary care provider. Of course, different combinations of fields may be displayed for each patient row by default or based upon a user's reconfiguration of the table 900.

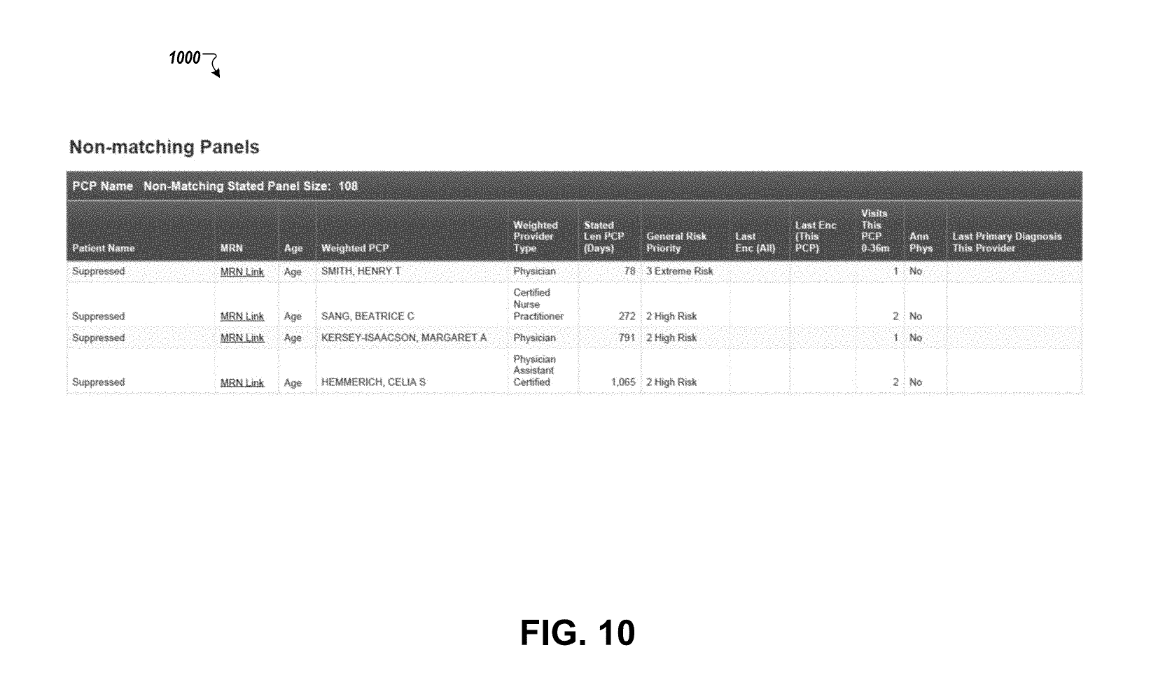

FIG. 10 shows a table 1000 for presentation in a graphical interface of a patient panel management system that identifies patients in a primary care provider's stated panel that are assigned to a different provider's weighted panel. The stated primary care provider may review this table 1000, for example, to determine whether to request re-assignment of the patients to the stated panels of the weighted providers. The table 1100 includes a row for each patient, where each row includes a field indicating the patient's name, a link to the electronic medical records file for the patient (MRN Link), an age indicator for the patient, the risk categorization for the patient (General Risk Priority), a date that the patient last saw the stated primary care provider, a number of visits to this primary care provider in the last 12 months and 36 months, whether the patient has had an annual physical in the last year, and the last primary diagnoses from the primary care provider. Of course, different combinations of fields may be displayed for each patient row by default or based upon a user's reconfiguration of the table 1100.

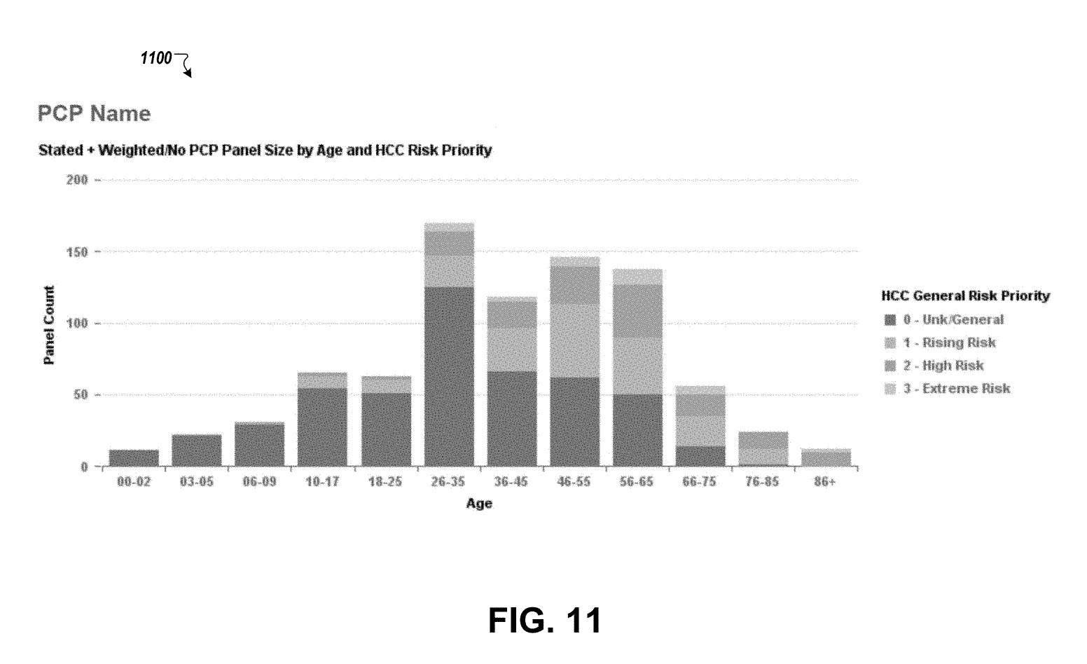

FIG. 11 shows a plot 1100 of the distribution of patients on a primary care provider's stated and weighted patient panels, organized by age and risk categorization (e.g., HCC risk priority). A primary care provider may access such a plot 1100 through the dashboard interface of the computing system in order to obtain a visual overview of his or her patient panels. The plot 1100 includes a respective bar for each of multiple patient age ranges. The height of the bars is proportional to the number of patients on the provider's panels in the corresponding age range. Further, each bar is visually formatted, such as by color coding, to indicate the proportion of patients in each age range that fall within each of the possible patient risk categories. Four risk categories represented by values in the range 0-3 are shown in the plot 1100 of FIG. 11. In some implementations, the primary care provider may adjust the parameters of the plot, e.g., by filtering to include only patients in the stated patient panel or weighted patient panel, adjusting the number of age ranges, or the granularity of risk categories.

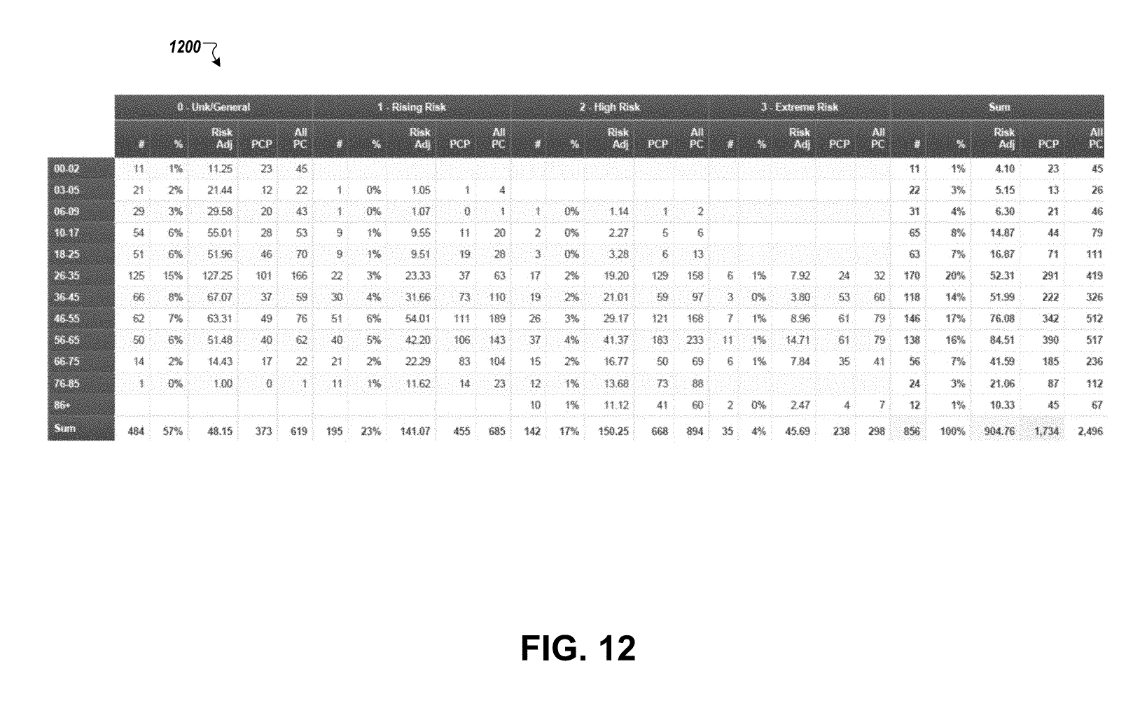

FIG. 12 shows a table 1200 for presentation in a graphical interface of a patient panel management system that shows the distribution of patients in a primary care provider's panel by age range and risk category.

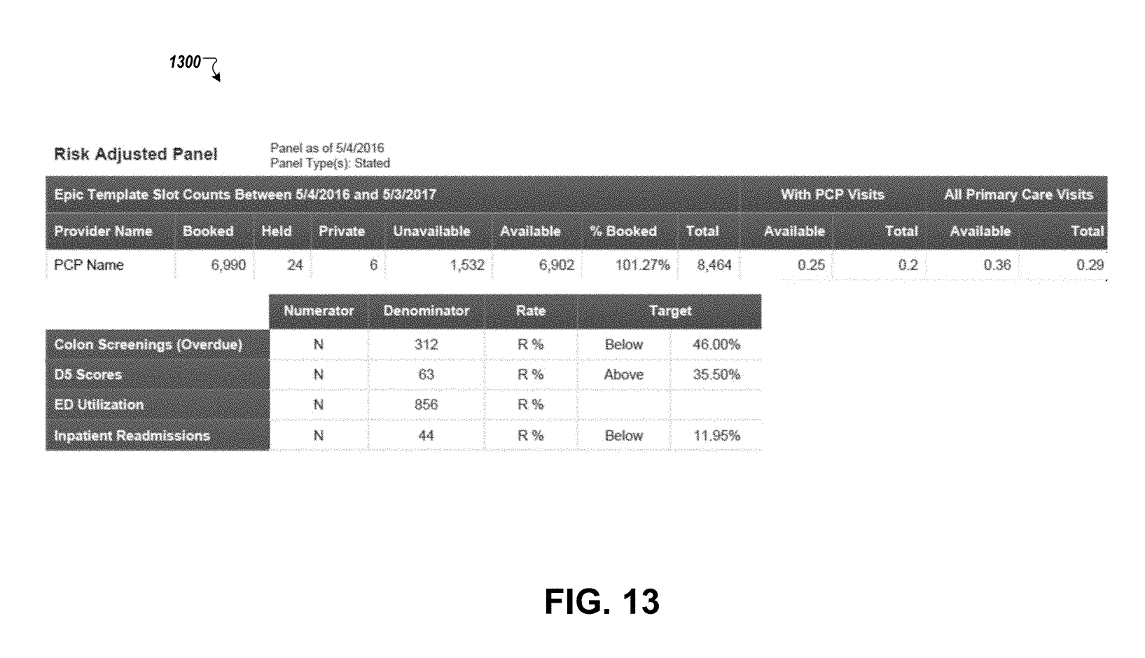

FIG. 13 shows a table 1300 for presentation in the graphical interface of a patient panel management system that shows a risk adjusted panel of a primary care provider.

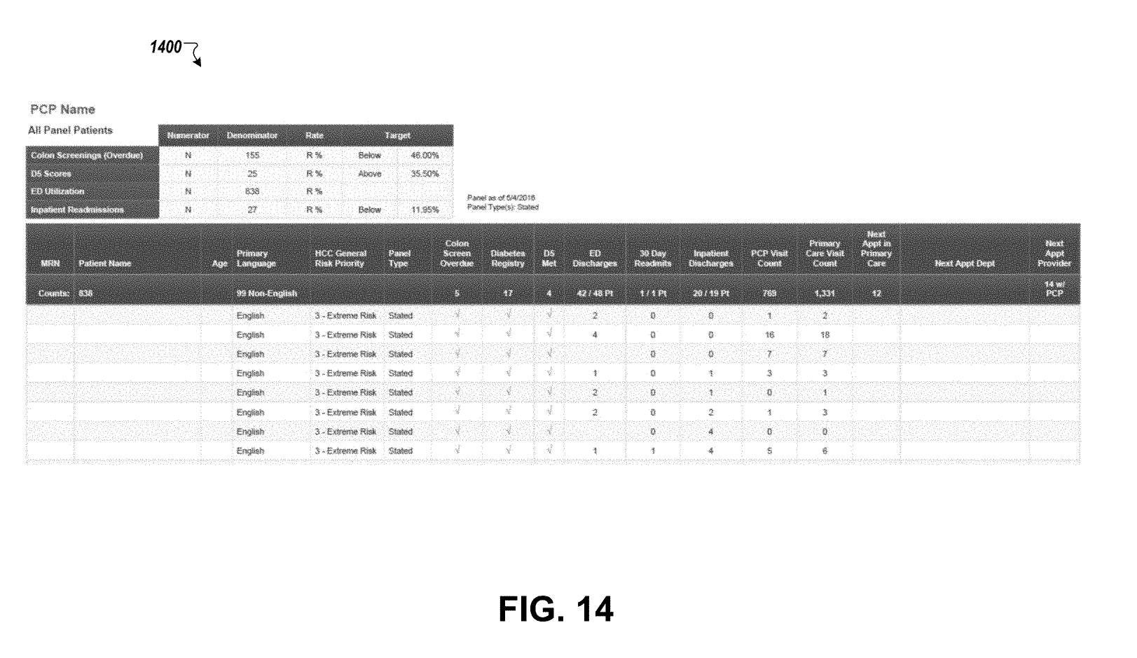

FIG. 14 shows a table 1400 for presentation in the graphical interface of the patient panel management system that shows all patients in a primary care provider's stated panel. Above the listing of patients, an overview is shown of metrics pertaining to the population of patients in the patient's panels. The metrics include, for example, information about the number of patients who are overdue on colon screenings, and the number of inpatient readmissions. The primary care provider or a supervisor, for instance, can review the summary information to identify the proportion of patients who are receiving recommended preventative services or are achieving other goals of the provider. In some implementations, a user can interact with the graphical interface to select a different subset of patients (e.g., the stated panel, weighted panel, matching panel, unweighted panel), and the summary information may be automatically refreshed to show metrics for the selected subset of patients.

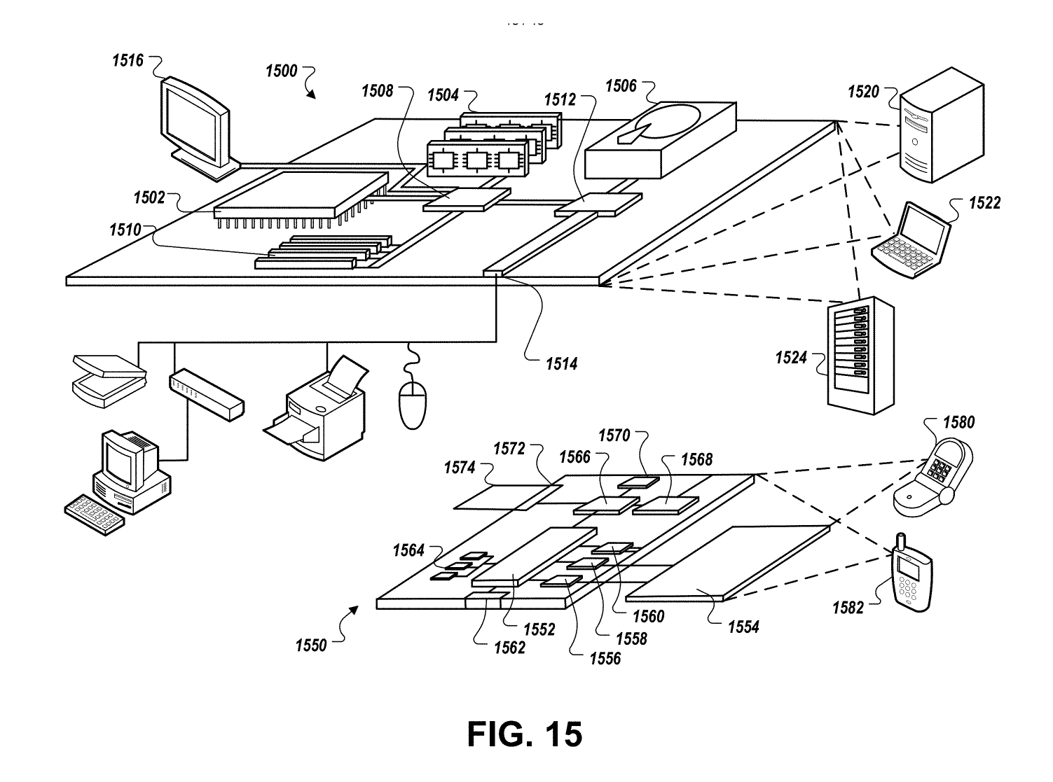

FIG. 15 shows an example of a computing device 1500 and a mobile computing device that can be used to implement the techniques described herein. The computing device 1500 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1500 includes a processor 1502, a memory 1504, a storage device 1506, a high-speed interface 1508 connecting to the memory 1504 and multiple high-speed expansion ports 1510, and a low-speed interface 1512 connecting to a low-speed expansion port 1514 and the storage device 1506. Each of the processor 1502, the memory 1504, the storage device 1506, the high-speed interface 1508, the high-speed expansion ports 1510, and the low-speed interface 1512, are interconnected using various busses, and may be mounted on a common motherboard or in other manners as appropriate. The processor 1502 can process instructions for execution within the computing device 1500, including instructions stored in the memory 1504 or on the storage device 1506 to display graphical information for a GUI on an external input/output device, such as a display 1516 coupled to the high-speed interface 1508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 1504 stores information within the computing device 1500. In some implementations, the memory 1504 is a volatile memory unit or units. In some implementations, the memory 1504 is a non-volatile memory unit or units. The memory 1504 may also be another form of computer-readable medium, such as a magnetic or optical disk.

The storage device 1506 is capable of providing mass storage for the computing device 1500. In some implementations, the storage device 1506 may be or contain a computer-readable medium, such as a floppy disk device, a hard disk device, an optical disk device, or a tape device, a flash memory or other similar solid state memory device, or an array of devices, including devices in a storage area network or other configurations. The computer program product may also contain instructions that, when executed, perform one or more methods, such as those described above. The computer program product can also be tangibly embodied in a computer- or machine-readable medium, such as the memory 1504, the storage device 1506, or memory on the processor 1502.

The high-speed interface 1508 manages bandwidth-intensive operations for the computing device 1500, while the low-speed interface 1512 manages lower bandwidth-intensive operations. Such allocation of functions is exemplary only. In some implementations, the high-speed interface 1508 is coupled to the memory 1504, the display 1516 (e.g., through a graphics processor or accelerator), and to the high-speed expansion ports 1510, which may accept various expansion cards (not shown). In the implementation, the low-speed interface 1512 is coupled to the storage device 1506 and the low-speed expansion port 1514. The low-speed expansion port 1514, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 1500 may be implemented in a number of different forms, as shown in the figure. For example, it may be implemented as a standard server 1520, or multiple times in a group of such servers. In addition, it may be implemented in a personal computer such as a laptop computer 1522. It may also be implemented as part of a rack server system 1524. Alternatively, components from the computing device 1500 may be combined with other components in a mobile device (not shown), such as a mobile computing device 1550. Each of such devices may contain one or more of the computing device 1500 and the mobile computing device 1550, and an entire system may be made up of multiple computing devices communicating with each other.

The mobile computing device 1550 includes a processor 1552, a memory 1564, an input/output device such as a display 1554, a communication interface 1566, and a transceiver 1568, among other components. The mobile computing device 1550 may also be provided with a storage device, such as a micro-drive or other device, to provide additional storage. Each of the processor 1552, the memory 1564, the display 1554, the communication interface 1566, and the transceiver 1568, are interconnected using various buses, and several of the components may be mounted on a common motherboard or in other manners as appropriate.

The processor 1552 can execute instructions within the mobile computing device 1550, including instructions stored in the memory 1564. The processor 1552 may be implemented as a chipset of chips that include separate and multiple analog and digital processors. The processor 1552 may provide, for example, for coordination of the other components of the mobile computing device 1550, such as control of user interfaces, applications run by the mobile computing device 1550, and wireless communication by the mobile computing device 1550.

The processor 1552 may communicate with a user through a control interface 1558 and a display interface 1556 coupled to the display 1554. The display 1554 may be, for example, a TFT (Thin-Film-Transistor Liquid Crystal Display) display or an OLED (Organic Light Emitting Diode) display, or other appropriate display technology. The display interface 1556 may comprise appropriate circuitry for driving the display 1554 to present graphical and other information to a user. The control interface 1558 may receive commands from a user and convert them for submission to the processor 1552. In addition, an external interface 1562 may provide communication with the processor 1552, so as to enable near area communication of the mobile computing device 1550 with other devices. The external interface 1562 may provide, for example, for wired communication in some implementations, or for wireless communication in other implementations, and multiple interfaces may also be used.

The memory 1564 stores information within the mobile computing device 1550. The memory 1564 can be implemented as one or more of a computer-readable medium or media, a volatile memory unit or units, or a non-volatile memory unit or units. An expansion memory 1574 may also be provided and connected to the mobile computing device 1550 through an expansion interface 1572, which may include, for example, a SIMM (Single In Line Memory Module) card interface. The expansion memory 1574 may provide extra storage space for the mobile computing device 1550, or may also store applications or other information for the mobile computing device 1550. Specifically, the expansion memory 1574 may include instructions to carry out or supplement the processes described above, and may include secure information also. Thus, for example, the expansion memory 1574 may be provide as a security module for the mobile computing device 1550, and may be programmed with instructions that permit secure use of the mobile computing device 1550. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. The computer program product contains instructions that, when executed, perform one or more methods, such as those described above. The computer program product can be a computer- or machine-readable medium, such as the memory 1564, the expansion memory 1574, or memory on the processor 1552. In some implementations, the computer program product can be received in a propagated signal, for example, over the transceiver 1568 or the external interface 1562.

The mobile computing device 1550 may communicate wirelessly through the communication interface 1566, which may include digital signal processing circuitry where necessary. The communication interface 1566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1568 using a radio-frequency. In addition, short-range communication may occur, such as using a Bluetooth, WiFi, or other such transceiver (not shown). In addition, a GPS (Global Positioning System) receiver module 1570 may provide additional navigation- and location-related wireless data to the mobile computing device 1550, which may be used as appropriate by applications running on the mobile computing device 1550.

The mobile computing device 1550 may also communicate audibly using an audio codec 1560, which may receive spoken information from a user and convert it to usable digital information. The audio codec 1560 may likewise generate audible sound for a user, such as through a speaker, e.g., in a handset of the mobile computing device 1550. Such sound may include sound from voice telephone calls, may include recorded sound (e.g., voice messages, music files, etc.) and may also include sound generated by applications operating on the mobile computing device 1550.

The mobile computing device 1550 may be implemented in a number of different forms, as shown in the figure. For example, it may be implemented as a cellular telephone 1580. It may also be implemented as part of a smart-phone 1582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine- readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In situations in which the systems, methods, devices, and other techniques here collect personal information (e.g., context data) about users, or may make use of personal information, the users may be provided with an opportunity to control whether programs or features collect user information (e.g., information about a user's social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

Although various implementations have been described in detail above, other modifications are possible. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

What is claimed is:

1. A computer-implemented method for empaneling primary care providers in a healthcare organization, the method comprising:
receiving, by a system of one or more computers and from a first client, a request for primary care panel information for a particular primary care provider (PCP) in the healthcare organization;
searching, by the system, a corpus of patient data that identifies a plurality of patients of the healthcare organization to determine a first set of patients for which the particular PCP is designated as a stated PCP for the patient, wherein the first set of patients form a stated patient panel for the particular PCP;
searching, by the system, the corpus of patient data to determine a second set of patients for which the particular PCP is designated as a weighted PCP for the patient,
wherein, for each patient in the second set of patients, the particular PCP is designated as the weighted PCP for the patient based on a quantity of interactions between the patient and the particular PCP during a past time interval with respect to quantities of interactions between the patient and other PCPs during the past time interval,
wherein the second set of patients form a weighted patient panel for the particular PCP,
generating, by the system, a response to the request for primary care information for the particular PCP, the response characterizing information about at least one of the stated patient panel for the particular PCP or the weighted patient panel for the particular PCP; and
providing the response to the first client to cause presentation of the information about the at least one of the stated patient panel for the particular PCP or the weighted patient panel for the particular PCP in a graphical interface on a display of the first client.

2. The method of claim 1, further comprising determining, by the system, a first panel score that is based on one or more characteristics of the stated patient panel for the particular PCP.

3. The method of claim 2, wherein the one or more characteristics of the stated patient panel from which the first panel score is determined include a size of the stated patient panel.

4. The method of claim 2, wherein the one or more characteristics of the stated patient panel from which the first panel score is determined include a risk profile of the stated patient panel, wherein the risk profile indicates an amount of healthcare services that are estimated to be consumed by patients in the stated patient panel.

5. The method of claim 1, further comprising determining, by the system, a second panel score that is based on one or more characteristics of the weighted patent panel for the particular PCP.

6. The method of claim 5, further comprising:
identifying that a value that is based on the first panel score for the stated patient panel and the second panel score for the weighted patient panel satisfies one or more criteria; and
in response to identifying that the value that is based on the first panel score for the stated patient panel and the second panel score for the weighted patient panel satisfies the one or more criteria, invoking a process to adjust the stated patient panel for the particular PCP.

7. The method of claim 6, wherein the value that is based on the first panel score for the stated patient panel and the second panel score for the weighted patient panel is a ratio or a difference of the first panel score and the second panel score, wherein the one or more criteria is a threshold ratio or a threshold difference, respectively.

8. The method of claim 6, wherein invoking the process to adjust the stated patient panel for the particular PCP comprises prompting the particular PCP to add a patient from the weighted patient panel to the stated patient panel.

9. The method of claim 2, wherein a level of compensation for the particular PCP is determined based at least in part on the first panel score for the stated patient panel.

10. The method of claim 1, further comprising determining, for each patient in at least a subset of the plurality of patients of the healthcare organization, a risk score that indicates an amount of healthcare services that are estimated to be consumed by patients in the stated patient panel.

11. The method of claim 10, wherein generating the response to the request for primary care information for the particular PCP comprises generating a report that lists patients in at least one of the stated patient panel or the weighted patient panel, wherein the listed patients in the report are sorted based at least in part on the risk scores for the listed patients.

12. The method of claim 10, wherein determining the risk score for a given patient in the at least the subset of the plurality of patients comprises determining a hierarchical condition category (HCC) score for the given patient.

13. The method of claim 10, wherein the risk score for a given patient in the at least the subset of the plurality of patients is determined based at least on two or more of demographic information for the given patient, health information for the given patient, and social determinant information for the given patient.

14. The method of claim 10, further comprising, for each patient in the at least the subset of the plurality of patients of the healthcare organization:
identifying a set of one or more characteristics for the patient; and
selecting, from a plurality of possible risk models, a particular risk model to apply to the patient; and
determining the risk score for the patient using the selected particular risk model.

15. The method of claim 14, wherein the set of one or more characteristics for the patient comprises at least one of age of the patient or a social determinant of the patient.

16. The method of claim 1, further comprising:
identifying a first patient as a result of the first patient being on the stated patient panel for the particular PCP but not being on the weighted patient panel for the particular PCP;
identifying a second PCP for which the first patient is on a weighted patient panel for the second PCP;
prompting the second PCP to add the first patient to a stated patient panel for the second PCP;
prompting the particular PCP to transfer the first patient to the stated patient panel for the second PCP; and
in response to (i) receiving an indication that the second PCP approves adding the first patient to the stated patient panel for the second PCP and (ii) receiving an indication that the particular PCP approves transferring the first patient to the stated patient panel for the second PCP, removing the first patient from the stated patient panel for the particular PCP and adding the first patient to the stated patient panel for the second PCP.

17. The method of claim 16, wherein prompting the second PCP to add the first patient to the stated patient panel comprises generating an alert for the second PCP that is accessible via a graphical interface of a patient empanelment dashboard, email, or a Short Message Service (SMS) message.

18. The method of claim 1, further comprising determining a value that indicates an amount of time that the particular PCP spends interacting with patients on the stated patient panel for the particular PCP.

19. The method of claim 18, further comprising:
determining a second value that indicates a total amount of time allotted to the particular PCP for interacting with patients; and
determining a third value that indicates a ratio of the value that indicates the amount of time that the particular PCP spends interacting with patients on the stated patient panel for the particular PCP and the second value that indicates a total amount of time allotted to the particular PCP for interacting with patients.

20. The method of claim 1, wherein the first client is a computing device that communicates with the system over a network.

21. The method of claim 1, wherein the first client is included among the one or more computers of the system.

22. The method of claim 1, wherein generating the response to the request for primary care information for the particular PCP comprises formatting an electronic document that includes the information about at least one of the stated patient panel for the particular PCP or the weighted patient panel for the particular PCP,
wherein providing the response to the first client comprises transmitting the formatted electronic document to the first client.

23. The method of claim 1, wherein generating the response to the request for primary care information for the particular PCP comprises generating an extensible markup language (XML) document that includes the information about at least one of the stated patient panel for the particular PCP or the weighted patient panel for the particular PCP,
wherein the first client is configured to format a second document for presentation in the graphical interface using the information from the XML document.

24. One or more non-transitory computer-readable media having instructions stored thereon that, when executed by one or more processors of a system, cause the one or more processors to perform operations comprising:
receiving, by the system and from a first client, a request for primary care panel information for a particular primary care provider (PCP) in the healthcare organization;
searching, by the system, a corpus of patient data that identifies a plurality of patients of the healthcare organization to determine a first set of patients for which the particular PCP is designated as a stated PCP for the patient, wherein the first set of patients form a stated patient panel for the particular PCP;
searching, by the system, the corpus of patient data to determine a second set of patients for which the particular PCP is designated as a weighted PCP for the patient,
wherein, for each patient in the second set of patients, the particular PCP is designated as the weighted PCP for the patient based on a quantity of interactions between the patient and the particular PCP during a past time interval with respect to quantities of interactions between the patient and other PCPs during the past time interval,
wherein the second set of patients form a weighted patient panel for the particular PCP,
generating, by the system, a response to the request for primary care information for the particular PCP, the response characterizing information about at least one of the stated patient panel for the particular PCP or the weighted patient panel for the particular PCP; and
providing the response to the first client to cause presentation of the information about the at least one of the stated patient panel for the particular PCP or the weighted patient panel for the particular PCP in a graphical interface on a display of the first client.

25. A computing system, comprising:
one or more processors; and
one or more computer-readable media having instructions stored thereon that, when executed by the one or more processors of a system, cause the one or more processors to perform operations comprising:
receiving, by the system and from a first client, a request for primary care panel information for a particular primary care provider (PCP) in the healthcare organization;
searching, by the system, a corpus of patient data that identifies a plurality of patients of the healthcare organization to determine a first set of patients for which the particular PCP is designated as a stated PCP for the patient, wherein the first set of patients form a stated patient panel for the particular PCP;
searching, by the system, the corpus of patient data to determine a second set of patients for which the particular PCP is designated as a weighted PCP for the patient,
wherein, for each patient in the second set of patients, the particular PCP is designated as the weighted PCP for the patient based on a quantity of interactions between the patient and the particular PCP during a past time interval with respect to quantities of interactions between the patient and other PCPs during the past time interval,
wherein the second set of patients form a weighted patient panel for the particular PCP,
generating, by the system, a response to the request for primary care information for the particular PCP, the response characterizing information about at least one of the stated patient panel for the particular PCP or the weighted patient panel for the particular PCP; and
providing the response to the first client to cause presentation of the information about the at least one of the stated patient panel for the particular PCP or the weighted patient panel for the particular PCP in a graphical interface on a display of the first client.
